(19) United States
(12) Patent Application Publication
(10) Pub. No.: US 2024/0299119 A1
(43) Pub. Date: Sep. 12, 2024

(54) SURGICAL HEAD RESTRAINT APPARATUS

(71) Applicant: I IDEAS LLC, St. James, NY (US)

(72) Inventors: Vincent Basilice, St. James, NY (US); Dahna Basilice, St. James, NY (US)

(21) Appl. No.: 18/597,767

(22) Filed: Mar. 6, 2024

Related U.S. Application Data
(60) Provisional application No. 63/450,442, filed on Mar. 7, 2023.

(51) Int. Cl. A61B 90/14

(52) U.S. Cl. CPC A61B 90/14

(57) ABSTRACT

A head restraint apparatus includes an elongate member extending along a longitudinal axis. The elongate member includes a central segment and opposed longitudinal end segments. The elongate member is configured for placement along a forehead of a subject and dimensioned to accommodate a length of medical tape positioned on the elongate member used to secure a head or neck area of the subject during a medical procedure. At least one set of vertical ribs extends from the elongate member. Individual vertical ribs of the at least one set are spaced to accommodate the length of medical tape therebetween.

### CROSS REFERENCE TO RELATED APPLICATION(S)

The present application claims the benefit of, and priority to, U.S. provisional Application Ser. No. 63/450,442, filed Mar. 7, 2023, the entire contents of which are incorporated by reference herein.

### BACKGROUND

### 1. Technical Field

The present invention relates to a medical or surgical apparatus, and, more particularly, relates to a medical apparatus for facilitating performance of a surgical procedure in the head or neck area of a subject. The present disclosure further relates to a head restraint apparatus for stabilizing the head and/or neck area of the subject during a surgical ophthalmic procedure. The present disclosure even further relates to an eye cover for use, for example, in association with the head restraint apparatus, in performing an ophthalmic procedure and/or in recovery.

### 2. Related Art

Surgical procedures involving the head or neck area of a subject require stabilizing the head relative to an object, for example, the head support of the operating table, to facilitate performance of the procedure. Conventional methodologies utilize surgical tape which is applied across the forehead of the subject and secured to the head rest of the operating table. This methodology presents several disadvantages including as follows: 1) the surgical tape irritates the skin during the procedure and particularly upon removal from the subject; and 2) in procedures involving the eye, removal of the tape typically causes the subject to close his eyes which is deleterious for a number of reasons including, for example, by compromising the wound resulting in wound leak and infection and dislocating an implant inserted during the procedure through the squeezing action of the eye lids.

Furthermore, in surgeries involving the eye, an eye cover is utilized for post operative care being preventive of touching or rubbing the eye while asleep. However, conventional eye covers have their own deficiencies including requirement of daily replacement of new eye covers and skin irritation along the attachment area of the eye cover around the eye.

### SUMMARY

In one illustrative embodiment, a head restraint apparatus includes an elongate member extending along a longitudinal axis. The elongate member includes a central segment and opposed longitudinal end segments. The elongate member is configured for placement along a forehead of a subject and dimensioned to accommodate a length of medical tape positioned on the elongate member used to secure a head or neck area of the subject during a medical procedure. At least one set of vertical ribs extends from the elongate member. Individual vertical ribs of the at least one set may be spaced to accommodate the length of medical tape therebetween.

In embodiments, the at least one set of vertical ribs is disposed adjacent at least one longitudinal end segment of the elongate member.

In other embodiments, first and second sets of vertical ribs are disposed adjacent respective longitudinal end segments of the elongate member. Each of the first and second sets of vertical ribs include the individual vertical ribs spaced to accommodate the length of medical tape therebetween.

In certain embodiments, the elongate member defines a curved contour to follow a contour of the forehead of the subject.

In embodiments, the elongate member includes a semi-rigid material whereby opposed longitudinal ends of the elongate member are spaced from the forehead of the subject when the length of medical tape is applied along the elongate member.

In other embodiments, at least the central segment of the elongate member is flexible to bend to follow a contour of the forehead of the subject.

In certain embodiments, the individual ribs of the at least one set of vertical ribs taper toward the central segment of the elongate member.

In embodiments, the elongate member includes an undersurface engageable with the forehead of the subject. The undersurface may include a substance to facilitate engagement with the forehead of the subject. The substance may comprise a tacky adhesive.

In another illustrative embodiment, a method comprises:

positioning an elongate member of a head restraint against a forehead of a subject where the subject is disposed on an operating table;

applying a length of medical tape along the elongate member; and

securing the medical tape relative to the operating table to stabilize a head or neck area of the subject.

In embodiments, applying the length of medical tape includes positioning the length of medical tape between at least one set of vertical ribs extending from the elongate member with individual vertical ribs of the at least one set of vertical ribs being spaced to accommodate the length of medical tape therebetween.

In certain embodiments, applying the length of medical tape includes positioning the length of medical tape between first and second sets of vertical ribs disposed adjacent respective longitudinal end segments of the elongate member. Each of the first and second sets of vertical ribs includes the individual vertical ribs spaced to accommodate the length of medical tape therebetween.

In other embodiments, positioning the elongate member includes applying a curved central segment of the elongate member against the forehead of the subject whereby the curved central segment following a contour of the forehead of the subject.

In embodiments, securing the medical tape includes spacing longitudinal ends of the elongate member relative to the forehead of the subject whereby the medical tape extending from the elongate member is spaced from the forehead of the subject.

### BRIEF DESCRIPTION OF THE DRAWINGS

The features of the application can be better understood with reference to the drawings described below, and the claims. The drawings are not necessarily to scale, emphasis instead generally being placed upon illustrating the principles described herein. In the drawings, like numerals are used to indicate like parts throughout the various views.

FIG. 1 is a first perspective view of the head restraint apparatus according in accordance with one or more illustrative embodiments of the present invention.

FIG. 2 is a second perspective view of the head restraint apparatus in accordance with one or more illustrative embodiments of the present invention.

FIG. 3 is a view illustrating application of the head restraint apparatus against the forehead of a subject in in accordance with one or more illustrative embodiments of the present invention.

FIG. 4 is a flow chart depicting a method of use of the head restraint apparatus in accordance with one or more illustrative embodiments of the present invention.

FIGS. 5 and 6 are perspective views of an eye shield for use with the head restraint apparatus in accordance with one or more illustrative embodiments of the present invention.

### DETAILED DESCRIPTION

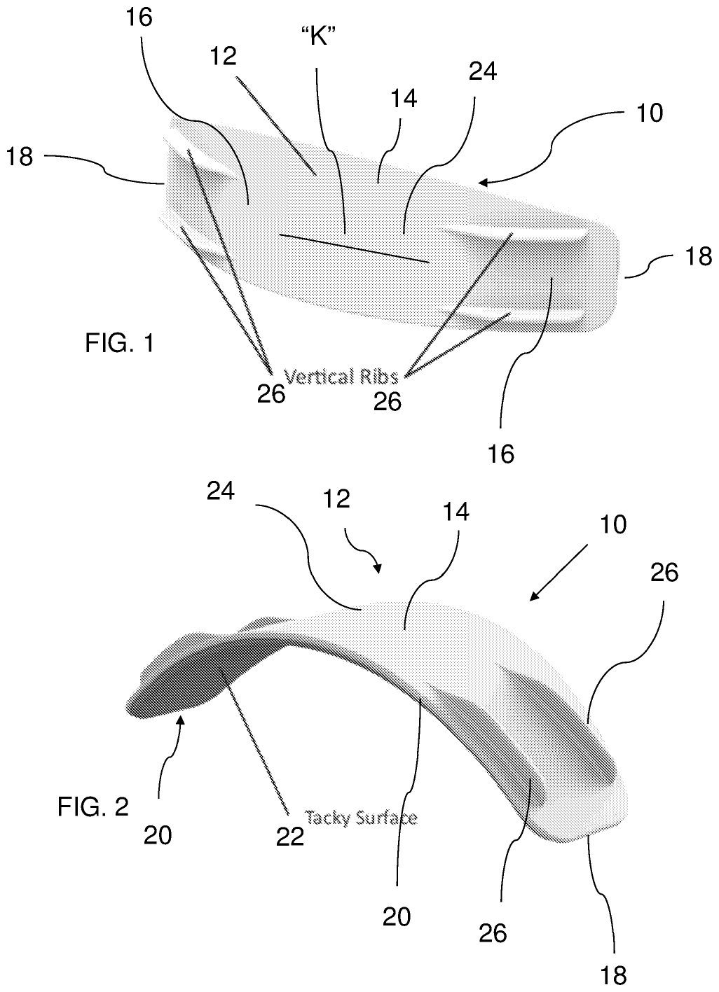

Referring now to FIGS. 1-3, the head restraint apparatus 10 according to the principles of the present invention is illustrated. The head restraint apparatus 10 is configured for use in a medical procedure such as a surgical ophthalmic procedure to restrain the head or neck area of a subject relative to an operating table. The head restraint apparatus 10 includes an elongate member 12 defining a longitudinal axis “k”. In illustrative embodiments, the elongate member 12 includes a central segment 14 and opposed longitudinal end segments 16 extending from the central segment 14. The opposed longitudinal end segments 16 extend to longitudinal ends 18. In illustrative embodiments, the elongate member 12 defines a curved contour at least along the central segment 14 and possibly the longitudinal end segments 16. The curved contour is configured to follow the contour of the forehead of a subject. In illustrative embodiments, the elongate member 12 is formed of a semi-rigid material and defines a curved contour in an unstressed normal condition of the elongate member 12. The semi-rigid material may comprise a polymeric material or a suitable metal material. The elongate member 12 may define a portion of a circle arranged about a central radius of curvature. The elongate member 12 includes an underside or contacting surface 20 which contacts the forehead of the subject. The contacting surface 20 may include a tacky surface, for example, a biocompatible adhesive 22 such as a polyurethane resin or the like to facilitate engagement with the skin of the forehead. Other biocompatible adhesives include an acrylate, silicon, silicon gel adhesives etc. or any other commercially available pressure sensitive adhesives having sufficient tack to form a tacky releasable bond to the skin

The elongate member 12 is dimensioned, in illustrative embodiments, to span between temple areas of the head and accommodate multiple sized heads. The elongate member may define a longitudinal length ranging from about two (2) inches to about twelve (12) inches or more and a width ranging from one-half (½) inch to three (3) inches or more. In some embodiments, multiple size elongate members 12 may be provided, including adult, child and infant sizes with respective dimensioning accordingly selected. For example, the longitudinal length may approximate the biocular breadth (e.g., distance between the outer corners of the eyes) or the bitragion breadth (e.g., the distance between the right and left tragions) of the fifty (50) percentile for an adult male, an adult female, child or infant.

The elongate member 12 includes an outer surface 24 opposing the contacting surface 20 upon which a length of medical tape “1” is applied. The outer surface 24 includes first and second sets of vertical ribs 26 along the longitudinal end segments 16 of the elongate member 12.

In illustrative embodiments, individual vertical ribs 26 of each set are spaced a predetermined distance sufficient to accommodate a length of medical tape “1” therebetween. The distance may range from about one-quarter inch (¼) inch to about two (2) inches. Other dimensions are also contemplated. In certain embodiments, the vertical ribs 26 taper toward the central segment 14. The tapering feature may facilitate positioning or application of the tape on the outer surface 22. In embodiments, the use of the term “vertical rib” is to include, without limitation, any element or feature extending from the outer surface 24. In embodiments, the term “vertical rib” may include any structure or element defined by a recess in the outer surface 24.

In certain illustrative embodiments, the elongate member 12 may be flexible to at least partially bend upon application to the forehead of the subject. In certain other embodiments, the elongate member 12 may be linear in a normal condition and bend upon application to the forehead of the subject. In embodiments, the elongate member 12 is configured to ensure that the longitudinal ends 18 of the elongate member 12 are spaced from the forehead of the subject whereby the medical tape does not engage any part of the forehead when applied to the forehead. This feature ensures that the length of medical tape “l” does not engage the forehead and thus avoids complications associated with conventional methodologies including skin irritation, squinting, etc. of eyes described herein above. In embodiments, the longitudinal ends 18 may have a different rigidity than the central segment 14 of the elongate member 12, for example, a greater rigidity than the central segment 14 to ensure the longitudinal ends 18 are spaced from the forehead upon application of the medical tape “t”. In some embodiments, the longitudinal ends 18 may have a greater thickness relative to the central segment 14 of the elongate member 12. In some embodiments, the presence or provision of the vertical ribs 26 provide a greater rigidity to the longitudinal ends 18 relative to the central segment 14 of the elongate member 12. In some embodiments, the elongate member 12 may have a similar flexibility throughout the length of the elongate member 12.

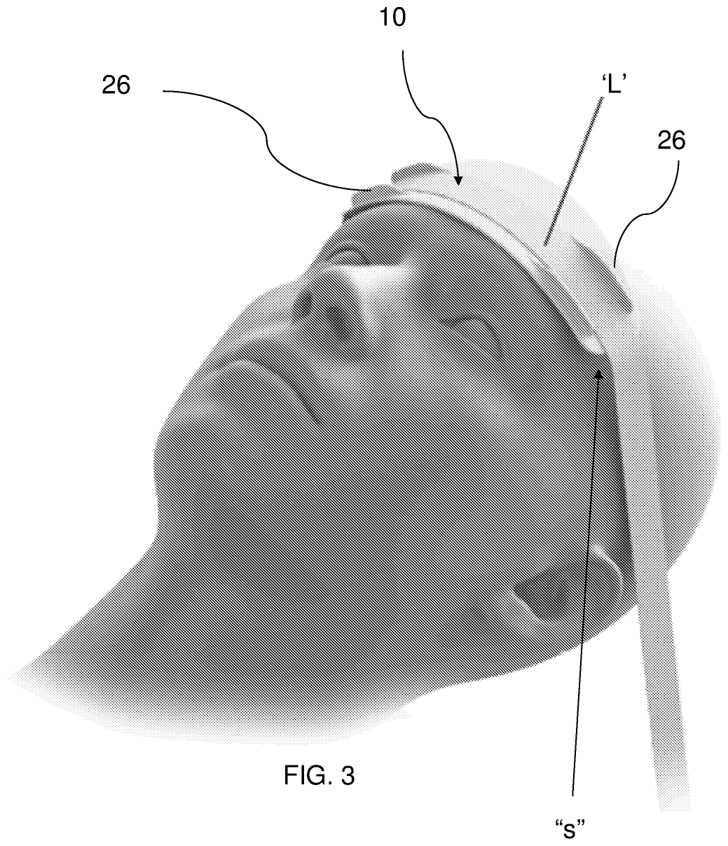

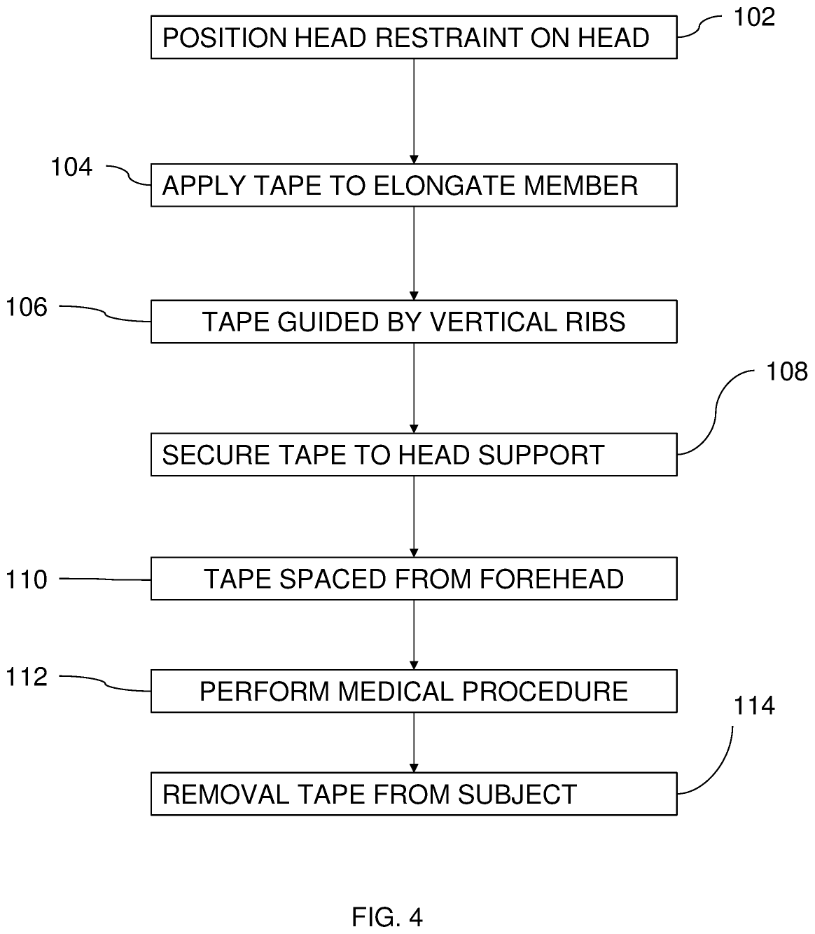

Referring now to FIG. 4, in conjunction with FIG. 3, a method of using the head restraint apparatus 10 will be discussed. The surgical process or method 100 includes positioning the elongate member 12 of the head restraint apparatus 10 on the forehead of the subject. (STEP 102). In illustrative embodiments, the contacting surface 20 includes a tack adhesive 22 to facilitate securing of the elongate member 12 on the forehead. In certain illustrative embodiments, the curved contour of the elongate member 12 follows the contour of the forehead of the subject. In other illustrative embodiments, the elongate member 12 flexes or bends to generally conform to the contour of the forehead. Once applied to the head, a length of medical tape “1” is applied onto the outer surface 22 of the elongate member 12. (STEP 104) Upon application to the outer surface, the sets of vertical ribs 26 guide the length of medical tape “1” onto the elongate member 12, and facilitate its retention between the vertical ribs 26 (STEP 106). The length of medical tape “1” is secured relative to the support, for example, the operating table or head support. (STEP 108) by wrapping around the back of the table headset. In the secured position of the length of medical tape “1”, the longitudinal ends 18 of the elongate member 12 are spaced “s” from the forehead, a defined spacing, to ensure that the medical tape “1” does not engage or come into contact with the forehead and head of the subject as depicted in FIG. 3. (STEP 110). One or more surgical procedures, including an ophthalmic procedure are performed on the subject (STEP 112). Upon completion of the surgery, the length of medical tape “l” is removed. (STEP 114). Due to the head restraint apparatus 10, the length of medical tape “1” is not peeled from the skin of the forehead thus avoiding complications associated therewith realized in conventional procedures.

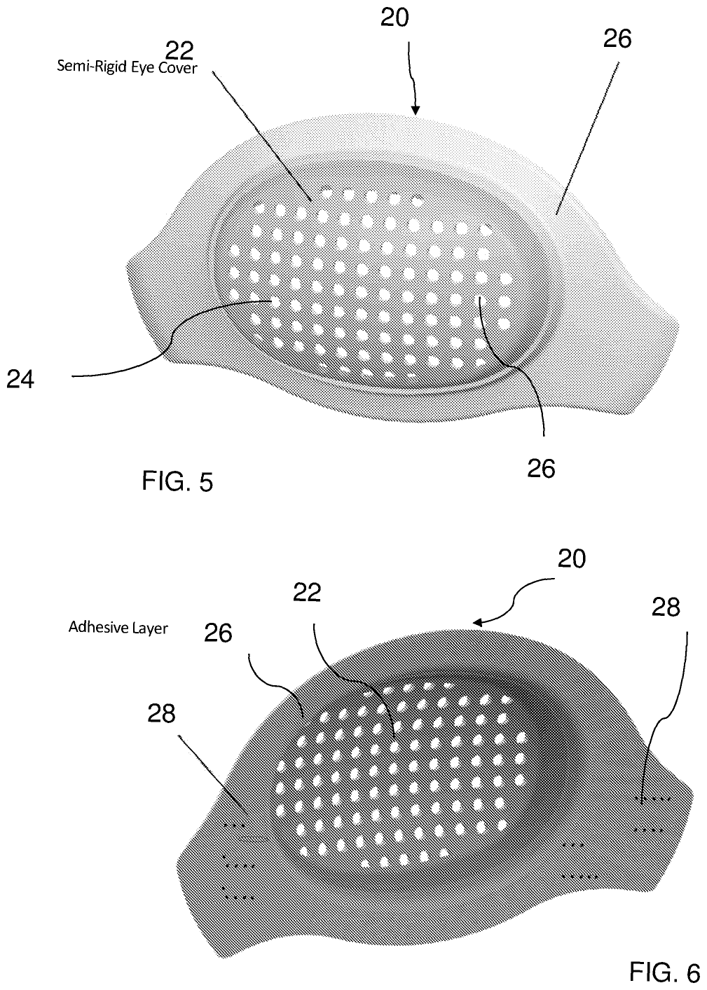

FIGS. 5 and 6 illustrate an eye shield apparatus 20 for use with the head restraint apparatus. The eye shield apparatus 20 is intended for use in eye surgery and post-operative care, and may be used in association with the head restraint apparatus 10. The eye care apparatus 20 includes a semi-rigid eye cover 22 with perforated holes 24 to allow visibility, and breathing, while limiting light exposure to the eye. A flexible shroud 26 is disposed about the periphery of the eye cover 22 and is configured to conform around the eye socket and not encroach the median plane of the face. The flexible shroud 26 includes wide flat surfaces configured to face flat along the skin surface around the eye socket, but not encroach over the median plane of the face and into the surgical field. An adhesive agent or layer 28 may be disposed on the flexible shroud 26 to contact the user's skin surface to adhere the eye cover over the protected eye for a continuous predetermined period of time for example up to ten (10) days or longer. The adhesive agent 28 may be any biocompatible adhesive agent including for example, synthetic rubber, acrylate, silicon, silicon gel adhesives etc. or any other commercially available pressure sensitive adhesives having sufficient tack to form a bond to the skin and application about the eye.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

While embodiments of the present disclosure have been particularly shown and described with reference to certain examples and features, it will be understood by one skilled in the art that various changes in detail may be effected therein without departing from the spirit and scope of the present disclosure as defined by claims that can be supported by the written description and drawings. Further, where exemplary embodiments are described with reference to a certain number of elements it will be understood that the exemplary embodiments can be practiced utilizing either less than or more than the certain number of elements.

What is claimed is:

1. A head restraint apparatus, which comprises:
an elongate member extending along a longitudinal axis, the elongate member including a central segment and opposed longitudinal end segments, the elongate member configured for placement along a forehead of a subject and dimensioned to accommodate a length of medical tape positioned on the elongate member used to secure a head or neck area of the subject during a medical procedure; and
at least one set of vertical ribs extending from the elongate member, individual vertical ribs of the at least one set of vertical ribs being spaced to accommodate the length of medical tape therebetween.

2. The head restraint apparatus according to claim 1 wherein the at least one set of vertical ribs is disposed adjacent at least one longitudinal end segment of the elongate member.

3. The head restraint apparatus according to claim 2 including:
first and second sets of vertical ribs disposed adjacent respective longitudinal end segments of the elongate member, each of the first and second sets of vertical ribs including the individual vertical ribs spaced to accommodate the length of medical tape therebetween.

4. The head restraint apparatus according to claim 1 wherein the elongate member defines a curved contour to follow a contour of the forehead of the subject.

5. The head restraint apparatus according to claim 4 wherein the elongate member comprises a semi-rigid material whereby opposed longitudinal ends of the elongate member are spaced from the forehead of the subject when the length of medial tape is applied along the elongate member.

6. The head restraint apparatus according to claim 1 wherein at least the central segment of the elongate member is flexible to bend to follow a contour of the forehead of the subject.

7. The head restraint apparatus according to claim 1 wherein the individual vertical ribs of the at least one set of vertical ribs taper toward the central segment of the elongate member.

8. The head restraint apparatus according to claim 1 wherein the elongate member includes an undersurface, the undersurface engaging the forehead of the subject, the undersurface including a substance to facilitate engagement with the forehead of the subject.

9. The head restraint apparatus according to claim 8 wherein the substance comprises a tacky adhesive.

10. A method comprising:
positioning an elongate member of a head restraint against a forehead of a subject, the subject being disposed on an operating table;
applying a length of medical tape along the elongate member; and
securing the medical tape relative to the operating table to stabilize a head or neck area of the subject.

11. The method according to claim 10 wherein applying the length of medical tape includes positioning the length of medical tape between at least one set of vertical ribs extending from the elongate member, individual vertical ribs of the at least one set of vertical ribs being spaced to accommodate the length of medical tape therebetween.

12. The method according to claim 11 wherein applying the length of medical tape includes positioning the length of medical tape between first and second sets of vertical ribs disposed adjacent respective longitudinal end segments of the elongate member, each of the first and second sets of vertical ribs including the individual vertical ribs spaced to accommodate the length of medical tape therebetween.

13. The method according to claim 10 wherein positioning the elongate member includes applying a curved central segment of the elongate member against the forehead of the subject, the curved central segment generally following a contour of the forehead of the subject.

14. The method according to claim 10 wherein securing the medical tape includes spacing longitudinal ends of the elongate member relative to the forehead of the subject whereby the medical tape extending from the elongate member is spaced from the forehead of the subject.
